(19) United States
(12) Patent Application Publication
(10) Pub. No.: US 2017/0306385 A1
(43) Pub. Date: Oct. 26, 2017

(54) METHODS FOR DISCOVERING THERAPEUTICS THAT ALTER THE STABILITY OF TARGET PROTEINS

(71) Applicant: Dana-Farber Cancer Institute, Inc., Boston, MA (US)

(72) Inventors: William G. Kaelin, Boston, MA (US); Gang Lu, San Diego, CA (US); Richard Middleton, Acton, MA (US); Kwok-kin Wong, Arlington, MA (US)

(73) Assignee: Dana-Farber Cancer Institute, Inc., Boston, MA (US)

(21) Appl. No.: 15/518,065

(22) PCT Filed: Oct. 9, 2015
(86) PCT No.: PCT/US15/54914

§ 371 Date: Apr. 10, 2017

Related U.S. Application Data
(60) Provisional application No. 62/062,257, filed on Oct. 10, 2014.

(51) Int. Cl. C12Q 1/02; G01N 33/68; G01N 33/50

(52) U.S. Cl. CPC C12Q 1/02; G01N 2333/00; G01N 33/5023; G01N 33/68

(57) ABSTRACT

The present application relates, in some aspects, to screening methods to identify test compounds that stabilize or destabilize a protein of interest. The present application is based, in some aspects, on the development of a plasmid that can be used to efficiently monitor the stabilities of thousands of proteins after specific perturbations. The plasmid allows for the co-expression of two reporter proteins, each of which is placed under the control of an IRES. In this way both reporters are transcribed together (i.e. are encoded by the same mRNA) and both are translated using an IRES.

### RELATED APPLICATIONS

This application claims the benefit under 35 U.S.C. §119(e) of U.S. provisional application U.S. Ser. No. 62/062,257, filed Oct. 10, 2014, the entire contents of which are incorporated herein by reference.

### FEDERALLY SPONSORED RESEARCH

This invention was made with government support under 2R01CA068490-19, and 2R01CA076120-13 awarded by National Institute of Health. The government has certain rights in the invention.

### BACKGROUND OF THE INVENTION

Reporter assays have been used routinely in the pharmaceutical and biotechnology industries to identify lead compounds that affect protein function. In the last decade, the chemist's ability to synthesize large numbers of chemical compounds in a short amount of time through techniques such as combinatorial chemistry has greatly increased, and often, thousands to millions of compounds need to be screened to identify those having a desired effect on a protein of interest.

Typically, reporter assays measure the activities of one reporter protein in a sample, but may combine multiple reporters. One strategy for co-expression of multiple reporters involves the design of bicistronic constructs, in which two genes separated by an internal ribosome entry site (IRES) sequence are expressed as a single transcriptional cassette (or bicistronic transcript) under the control of a common upstream promoter (Yen et al., Science. 2008 Nov. 7; 322(5903):918-23). The intervening IRES sequence functions as a ribosome-binding site for efficient cap-independent internal initiation of translation. Such a design enables transcription of both genes with IRES-directed cap-independent translation. This system allows for co-expression of both a control reporter, not expected to change upon experimental treatment, along with a test reporter that is normalized to the control in each test sample. However, many perturbations in the cell can differentially affect cap-dependent translation compared to cap-independent translation. Moreover, some IRESes have been shown to display variable expression of the downstream gene (Wong et al. Gene Ther. 2002 March; 9(5):337-44). This leads to high false positives and unreliable reporter assays. Thus, there is a need for an efficient high-throughput approach for analysis of protein stability where nonspecific alterations in reporter activity are used to control for the inherent variability in cell based protein stability assays. This allows for reducing the error in the data required to effectively and efficiently run an HTS assay.

### SUMMARY OF THE INVENTION

The present disclosure relates, in some aspects, to the development of a plasmid that can be used to efficiently monitor the stabilities of thousands of proteins after specific perturbations.

According to some aspects, the present disclosure provides a method to identify a test compound that stabilizes or destabilizes a protein of interest, the method comprising:
(i) contacting a transformed host cell comprising a DNA plasmid with a test compound, wherein the plasmid comprises in operable linkage
   (a) a promoter,
   (b) a first internal ribosomal entry site (IRES);
   (c) a nucleotide sequence encoding a first reporter protein;
   (d) a second IRES; and
   (e) a nucleotide sequence encoding a second reporter protein,
   wherein an open reading frame (ORF) is fused to the nucleotide sequence encoding a first reporter protein or to the nucleotide sequence encoding a second reporter protein and wherein said open reading frame codes for a protein of interest;
(ii) determining ratios of fused reporter protein signal to unfused reporter protein signal in presence and absence of the test compound; and
(iii) identifying said test compound as a stabilizer when the ratio of fused reporter protein signal to unfused reporter protein signal in the presence of the test compound is increased as compared to the ratio of fused reporter protein signal to unfused reporter protein signal in the absence of the test compound, and identifying said test compound as a destabilizer when the ratio of fused reporter protein signal to unfused reporter protein signal in the presence of the test compound is decreased as compared to the ratio of fused reporter protein signal to unfused reporter protein signal in the absence of the test compound.

In some embodiments, the first and second reporter proteins have distinguishable detectable reporter signals. In some embodiments, the first and second reporter proteins are enzyme proteins having distinguishable signals generated from their products. In some embodiments, the first and second reporter proteins are bioluminescent proteins having distinguishable bioluminescence signals. In some embodiments, the first and second reporter proteins are fluorescent proteins having distinguishable fluorescence signals. In some embodiments, the first and second reporter proteins are selected from the group consisting of renilla luciferase (Rluc) and firefly luciferase (FLuc). In some embodiments, the first and second reporter proteins are selected from the group consisting of green fluorescence protein and red fluorescence protein. In some embodiments, the promoter is a eukaryotic promoter or a synthetic promoter. In some embodiments, the promoter comprises cytomegalovirus (CMV) promoter. In some embodiments, the open reading frame is derived from an ORFeome of an organism. In some embodiments, the open reading frame encodes an oncoprotein. In some embodiments, the oncoprotein is selected from the group consisting of MYC, Ikaros family zinc finger protein 1 (IKZF1), Ikaros family zinc finger protein 3 (IKZF3), Interferon regulatory factor 4 (IRF4), mutant p53, N-Ras, c-Fos, and c-Jun. In some embodiments, contacting a transformed host cell comprising the plasmid with a test compound comprises growing the transformed host cell in the presence of the test compound for an appropriate time.

Each of the embodiments and aspects of the invention can be practiced independently or combined. Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

These and other aspects of the inventions, as well as various advantages and utilities will be apparent with reference to the Detailed Description. Each aspect of the invention can encompass various embodiments as will be understood.

All documents identified in this application are incorporated in their entirety herein by reference.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 confirms that pIRIGF constructs express in 293FT and HELA cells (FIG. 1A-C) and pUG-FIRP constructs express in U-2 OS cells (FIG. 1D). Several different versions of mammalian and lentiviral plasmid constructs were tested for their ability to generate cells (e.g. 293FT, HELA, or U-2 OS cells) expressing tagged target proteins (e.g., firefly or NanoLuc tag) and co-expressing a reporter luciferase (e.g., Renilla or Firefly).

In FIG. 2, 293FT and HELA cells were transfected with IKZF1-firefly, IKZF3-firefly and MYC-firefly fusion proteins and selected using puromycin and geneticin respectively. These pools were very unstable and lost signals in 10 to 30 days and generally had very small responses to IMiD's (FIG. 2A-C). Therefore, individual clones were isolated using a limited cloning strategy in 96-well plates. Surviving cells were isolated as colonies, further expanded and tested for luciferase signals and response to IMiDs. Clone 2B4 was identified as a strong responder to lenalidomide. HELA cells expressed very low levels of luciferase making isolation of HELA clones very difficult. Detection by western blots of firefly, IKZF1 and myc confirmed expression of the fusion protein and relative expression correlated with firefly luciferase signals (FIG. 2D).

In FIG. 3 cell line clones (IKZF1-2B4, IKZF1-2B11, myc-1C3 and myc-5F2) expressing the indicated firefly fusion protein were evaluated in the dual-glo assay for reproducibility. Potency of IMiD's and relative reduction in firefly luciferase signals confirmed the expected responses and generated data with Z′ values sufficient for screening (FIG. 3A-D).

FIG. 4 shows pilot screen results for IKZF1 2B4 cells—Active compounds (Prestwick collection; FIG. 4A) and NCI collection; FIG. 4B).

FIG. 5 shows pilot screen results for MYC 5F2 cells—Active compounds NCI collection).

FIG. 6 confirms the hits tested on IKZF1 2B4 and MYC 5F2 cell lines (FIG. 6A-C). Summary retest data from commercial compounds and from DTP compounds is shown in FIG. 6D-E.

FIG. 7 shows confirmation data using Western blots. IKZF1 2B4 cell line examples (FIG. 7A-B), MYC 5F2 example (FIG. 7C)

FIG. 8 shows further evaluations of HSP90 inhibitors. FIG. 8A demonstrates testing of HSP 90 inhibitors CCT018159 and geldanamycin on cells transiently transfected with the MYC-firefly fusion protein. FIG. 8B shows testing of HSP 90 inhibitors CCT018159 and geldanamycin on 293FT cells stably expressing MYC-firefly fusion protein. FIG. 8C shows testing of several HSP-90 inhibitors at various doses on 5 different cell lines stably expressing the MYC-firefly fusions protein. FIG. 8D compares the HSP90 inhibitor BIIB021 and pomalidomide on 293FT cells transiently expressing IKZF1-firefly fusion protein.

FIG. 9 shows an overview of the ICCB screening results. Specifically, it shows cherry pick retests for IKZF1 ICCB screen.

FIG. 10 shows compares activity in IKZF1 vs. MYC cell lines. 133 cherry picks in IKZF1 and MYC cell lines were tested.

FIG. 11 shows a better dose response at 16 hours for HSP90 inhibitors: BIIB021 (FIG. 11A) and PF-04929113 (FIG. 11B).

FIG. 12 shows cyclohexamide time course on 7 MYC cell lines including cyclohexamide untagged luciferase (FIG. 12A) and cyclohexamide tagged luciferase (FIG. 12B).

FIG. 13 shows MG132 time course on 7 MYC cell lines including MG-132 tagged luciferase (FIG. 13A) and MG-132 untagged luciferase (FIG. 13B).

FIG. 14 shows MLN4924 time course on 7 MYC cell lines including MLN4924 tagged luciferase (FIG. 14A) and MLN4924 untagged luciferase (FIG. 14B).

FIG. 15 shows A549-MYC-firefly & H1299-MYC-firefly Western Blot confirmation after MYC knockdown using 48 hour treatment with siRNA directed to MYC mRNA. The reduction in fusion protein, as observed by western blotting with MYC and firefly directed antibodies (FIG. 15A), is comparted to the decrease in luciferase signals (FIG. 15B). MYC antibody also detects the decrease in endogenous MYC.

FIG. 16 shows screening results from a commercial library of siRNA's directed to the family of DUB enzymes including A549 (FIG. 16A), H1299 (FIG. 16B), and HEK293T (FIG. 16C) cells expressing MYC-firefly and U2OS (FIG. 16D) cells expressing MYC-nanoluc.

### DETAILED DESCRIPTION OF THE INVENTION

The present application is based, in some aspects, on the development of a plasmid that can be used to efficiently monitor the stabilities of thousands of proteins after specific perturbations. The plasmid allows for the co-expression of two reporter proteins, each of which is placed under the control of an IRES. In this way both reporters are transcribed together (i.e. are encoded by the same mRNA) and both are translated using an IRES. This minimizes the problem of spurious changes in the ratio of the two reporters caused by perturbations (e.g. compounds) that differentially effect IRES-dependent versus IRES-independent translation, and thus minimizes false positives.

According to some aspects, the present disclosure provides a method to identify a test compound that stabilizes or destabilizes a protein of interest. The method comprises

(i) contacting a transformed host cell comprising a DNA plasmid with a test compound, wherein the plasmid comprises in operable linkage

(a) a promoter,

(b) a first internal ribosomal entry site (IRES);

(c) a nucleotide sequence encoding a first reporter protein;

(d) a second IRES; and

(e) a nucleotide sequence encoding a second reporter protein,

wherein an open reading frame (ORF) is fused to the nucleotide sequence encoding a first reporter protein or to the nucleotide sequence encoding a second reporter protein and wherein said open reading frame codes for a protein of interest;

(ii) determining ratios of fused reporter protein signal to unfused reporter protein signal in presence and absence of the test compound; and

(iii) identifying said test compound as a stabilizer when the ratio of fused reporter protein signal to unfused reporter protein signal in the presence of the test compound is increased as compared to the ratio of fused reporter protein signal to unfused reporter protein signal in the absence of the test compound, and identifying said test compound as a destabilizer when the ratio of fused reporter protein signal to unfused reporter protein signal in the presence of the test compound is decreased as compared to the ratio of fused reporter protein signal to unfused reporter protein signal in the absence of the test compound.

As used herein, “operable linkage” refers to a functional linkage between two nucleic acid sequences, such as a transcription control element (e.g., a promoter) and the linked transcribed sequence. Thus, a promoter is in operable linkage with a gene if it can mediate transcription of the gene.

As used herein a “promoter” usually contains specific DNA sequences (responsive elements) that provide binding sites for RNA polymerase and transcriptional factors for transcription to take place. In some embodiments, the promoter is a eukaryotic promoter or a synthetic promoter. Examples of promoters include, but are not limited to, the TATA box, the SV40 late promoter from simian virus 40, cytomegalovirus (CMV) promoter, ubiquitin C promoter (UbC promoter) and the T7 promoter. These and other promoter sequences are well known in the art. In one example of the invention, the promoter is a CMV promoter. In one example of the invention, the promoter is a UbC promoter.

As used herein, an “internal ribosomal entry site” or “IRES” is a cis acting nucleic acid element that mediates the internal entry of ribosomes on an RNA molecule and thereby regulates translation in eukaryotic systems. In the methods and compositions of the present invention, a first and a second IRES elements are contained in the plasmid. The first and second IRES elements permit the independent translation of a nucleotide sequence encoding a reporter protein and an open reading frame fused to a nucleotide sequence encoding another reporter protein from a single messenger RNA. In some embodiments, the first and second IRESs are the same (i.e., they have identical sequences). In some embodiments, the first and second IRESs are not the same (i.e., they do not have identical sequences).

Many IRES elements have been identified in both viral and eukaryotic genomes. In addition, synthetic IRES elements have also been developed. For example, IRES elements have been found in a variety of viruses including members of the genus Enterovirus (e.g. human poliovirus 1 (Ishii et al. (1998) J Virol. 72:2398-405 and Shiroki et al. (1997) J. Virol. 77:1-8), human Coxsackievirus B); Rhinovirus (e.g., human rhinovirus); Hepatovirus (Hepatitis A virus); Cardiovirus (Encephalomyocarditis virus ECMV (nucleotides 2137-2752 of GenBank Accession No. AB041927 and Kim et al. (1992) Mol Cell Biology 72:3636-43) and Etheirler's encephalomyelitis virus); Aphtovirus (Foot- and mouth disease virus (nucleotides 600-1058 of GenBank Accession No. AF308157; Belsham et al. (1990) EMBO 77:1105-10; Poyry et al. (2001) RNA 7:647-60; and Stoneley et al. (2000) Nucleic Acid Research 25:687-94), equine rhinitis A virus, Ewuine rhinitis B); Pestivirus (e.g., Bovine viral diarrhea virus (Poole et al. (1995) Virology 206:150-154) and Classical swine fever virus (Rijnbrand et al. (1997) J. Virol 77:451-7); Hepacivirus (e.g., Hepatitis C virus (Tsukiyama-Kohara et al. (1992) J. Virol. 66:1476-1483, Lemon et al. (1997) Semin. Virol. 5:274-288, and nucleotide 1201-1812 of GenBank Accession No. AJ242654.) and GB virus B). Each of these references is herein incorporated by reference.

IRES elements have also been found in viruses from the family Retroviridae, including members of the Lentivirus family (e.g., Simian immunodeficiency virus (Ohlmann et al. (2000) Journal of Biological Chemistry 275:11899-906) and human immunodeficiency virus 1 (Buck et s/. (2001) J Virol. 75:181-91); the BLV-HTLV retroviruses (e.g., Human T-lymphotrophic virus type 1 (Attal et al. (1996) EEES Letters 392:220-4); and the Mammalian type C retoviral family (e.g., Moloney murine leukemia virus (Vagner et al. (1995) J. Biol. Chem 270:20316-83), Friend murine leukemia virus, Harvey murine sarcoma virus, Avian retriculoendotheliosis virus (Lopez-Lastra et al. (1997) Hum. Gene Ther 5:1855-65), Murine leukemia virus (env RNA) (Deffaud et al. (2000) J. Virol. 74:846-50), Rous sarcoma virus (Deffaud et al. (2000) J. Virol. 74:11581-8). Each of these references is herein incorporated by reference.

Eukaryotic mRNAs also contain IRES elements including, for example, BiP (Macejak et al. (1991) Nature 355:91); Antennapedia of Drosophilia (exons d and e) (Oh et al. (1992) Genes and Development 6:1643-1653; c-myc; and, the X-linked inhibitor of apoptosis (XIAP) gene (U.S. Pat. No. 6,171,821).

Various synthetic IRES elements have been generated. See, for example, De Gregorio et al. (1999) EMBO J. 75:4865-74; Owens et al. (2001) PNAS 4:1471-6; and Venkatesan et al. (2001) Molecular and Cellular Biology 21:2826-37. For additional IRES elements known in the art, see, for example, rangueil.inserm.fr/IRESdatabase.

In a specific embodiment, the IRES sequence is derived from encephalomyocarditis virus (ECMV).

As used herein, a reporter protein is any protein that can be specifically detected when expressed (i.e, has a detectable signal when expressed), for example, via its fluorescence or enzyme activity. The plasmid comprises a nucleotide sequence encoding a first reporter protein and a nucleotide sequence encoding a second reporter protein. An open reading frame is fused either to the nucleotide sequence encoding a first reporter protein or to the nucleotide sequence encoding a second reporter protein. In some embodiments, the open reading frame is fused to the nucleotide sequence encoding a first reporter protein. In some embodiments, the open reading frame is fused to the nucleotide sequence encoding a second reporter protein. This allows one to study the expression of the linked open reading frame in response to different stimuli. As used herein, “fused” is intended to mean that the amino acids encoded by the ORF and the reporter protein are joined by peptide bonds to create a contiguous protein sequence. Thus, the reporter protein fused to the open reading frame serves as a marker of the stability of the fused open reading frame. The other reporter protein that is unfused to the open reading frame (and thus does not create a contiguous protein sequence with the amino acids encoded by the ORF) serves as an internal control to normalize for cell number and expression variability.

Typically, the first and second reporter proteins have distinguishable detectable reporter signals. For example, the first and second reporter proteins are enzyme proteins having distinguishable signals generated from their products. In some embodiments, the first and second reporter proteins are bioluminescent proteins that emit light at different wavelengths and/or utilize different substrates. Alternatively, the first and second reporter proteins are fluorescent proteins that fluoresce at different wavelengths.

Many reporter proteins known in the art may be used, including but not limited to bioluminescent proteins, fluorescent reporter proteins, and enzyme proteins such as beta-galactosidase, horse radish peroxidase and alkaline phosphatase that produce specific detectable products. The fluorescent reporter proteins include, for example, green fluorescent protein (GFP), cyan fluorescent protein (CFP), red fluorescent protein (RFP) and yellow fluorescent protein (YFP) as well as modified forms thereof e.g. enhanced GFP (EGFP), enhanced CFP (ECFP), enhanced RFP (ERFP), mCHERRY, and enhanced YEP (EYEP).

Examples of bioluminescent proteins, such as luciferases, including but not limited to renilla luciferase (Rluc), firefly luciferase (FLuc) and NanoLuc, are known in the art (see, for example, Fan, F. and Wood, K., Assay and drug development technologies V5 #1 (2007); Gupta, R. et al Nature Methods V8 #10 (2011); Nano-Glo® Luciferase Assay System (Promega) and en.wikipedia.org/wiki/Bioluminescence.

Other non-limiting examples of reporter proteins are shown below:

Species-specific luciferase specificity, cofactor requirements and physical characteristics.

| | | Size | | | |
|---|---|---|---|---|---|
| Organism | Luciferase | (kDa) | Substrate | Requires | Secreted |
| Photinus pyralis | North American firefly | 61 | D-luciferin | Mg, ATP | No |
| | luciferase | | | | |
| Luciola cruciata | Japanese firefly (Genji-botaru) | 64 | D-luciferin | Mg, ATP | No |
| | luciferase | | | | |
| Luciola italica | Italian firefly Luciferase | 64 | D-luciferin | Mg, ATP | No |
| Luciola lateralis | Japanese firefly (Heike) | 64 | D-luciferin | Mg, ATP | No |
| | luciferase | | | | |
| Luciola mingrelica | East European firefly luciferase | 64 | D-luciferin | Mg, ATP | No |
| Photuris pennsylvanica | Pennsylvania firefly luciferase | 64 | D-luciferin | Mg, ATP | No |
| Pyrophorus plagiophthalamus | Click beetle luciferase | 64 | D-luciferin | Mg, ATP | No |
| Phrixothrix hirtus | Railroad worm luciferase | 64 | D-luciferin | Mg, ATP | No |
| Renilla reniformis | Renilla luciferase | 36 | Coelenterazine | N/A | No |
| | Rluc8 (mutant of Renilla | 36 | Coelenterazine | N/A | No |
| | luciferase | | | | |
| | Green Renilla luciferase | 36 | Coelenterazine | N/A | No |
| Gaussia princeps | Gaussia luciferase | 20 | Coelenterazine | N/A | Yes |
| | Gaussia-Dura luciferase | 20 | Coelenterazine | N/A | Yes |
| Cypridina noctiluca | Cypridina luciferase | 62 | Vargulin/Cypridina | N/A | Yes |
| | | | luciferin | | |
| Cypridina hilgendorfii | Cypridina (Vargula) luciferase | 62 | Vargulin/Cypridina | N/A | Yes |
| | | | luciferin | | |
| Metridia longa | Metridia luciferase | 23.8 | Coelenterazine | N/A | Yes |
| Oplophorus gracilorostris | OLuc | 19 | Coelenterazine | N/A | Yes |

In some embodiments, the first and second reporter proteins are selected from the group consisting of renilla luciferase (Rluc), firefly luciferase (FLuc) and NanoLuc. In some embodiments, the first and second reporter proteins are selected from the group consisting of green fluorescence protein and red fluorescence protein.

An open reading frame is fused either to the nucleotide sequence encoding a first reporter protein or to the nucleotide sequence encoding a second reporter protein. The open reading frame is fused to the 5′ or to the 3′ end of the nucleotide sequence. As used herein, an open reading frame or ORF refers to a sequence of nucleotides that codes for a contiguous sequence of amino acids. The translated open reading frame may be all or a portion of a gene encoding a protein or polypeptide of interest.

The ORF of the plasmid codes for a protein of interest. As used herein, a “protein of interest” can be any conceivable polypeptide or protein that may be of interest, such as to study or otherwise characterize. In some embodiments, the ORF may be derived from an ORFeome of an organism. A complete ORFeome contains nucleic acids that encode all proteins of a given organism. A representative fraction of a full ORFeome is at least 60% of all proteins expressed by the organism. In some embodiments, the organism is a mammal. In some embodiments, the mammal is human.

In some embodiments, the protein of interest is a human polypeptide or protein. In some embodiments, the protein of interest is an oncoprotein, such as, but not limited to, RAS, MYC, SRC, FOS, JUN, MYB, ABL, BCL2, HOX11, HOX11L2, TAL1/SCL, LMO1, LM02, EGFR, MYCN, MDM2, CDK4, GLI1, IGF2, EGFR, FLT3-ITD, TP53, PAX3, PAX7, BCR/ABL, HER2 NEU, FLT3R, FLT3-ITD, TAN1, B-RAF, E2A-PBX1, and NPM-ALK, as well as fusion of members of the PAX and FKHR gene families, WNT, MYC, ERK EGFR, FGFR3, CDH5, KIT, RET, Interferon regulatory factor 4 (IRF4) and TRK. Other exemplary oncogenes are well known in the art and several such examples are described in, for example, The Genetic Basis of Human Cancer (Vogelstein, B. and Kinzler, K. W. eds. McGraw-Hill, New York, N.Y., 1998).

In some embodiments, the protein of interest is a transcription factor. Some examples of such transcription factors include (but are not limited to) the STAT family (STATs 1, 2, 3, 4, 5a, 5b, and 6), FOS/JUN, NF κB, HIV-TAT, and the E2F family. In some embodiments, the protein of interest is an IKAROS family zinc finger protein. In some embodiments, the protein of interest is IKZF1, IKZF2, IKZF3, IKZF4, or IKZF5. In some embodiments, the protein of interest is IKZF1 or IKZF3.

The nucleotide sequence encoding a reporter protein and the fused ORF are “in frame”, i.e., consecutive triplet codons of a single polynucleotide comprising the nucleotide sequence encoding the reporter protein and the fused open reading frame encode a single continuous amino acid sequence.

The methods described herein allows one to screen libraries of compounds and identify a test compound that stabilizes or destabilizes a protein of interest. A compound library is a collection of stored compounds typically used in high-throughput screening. The library compounds may include, for example, synthesized organic molecules, naturally occurring organic molecules, peptides, polypeptides, nucleic acid molecules and components thereof. Examples of compound library include, but are not limited to, Screen-Well® Compound Libraries (Enzo Life Sciences), EXPRESS-Pick Collection and CORE Library (Chem Bridge), National Cancer Institute Library, Prestwick Chemical Library® and Tocriscreen Compound Library Collections.

The plasmids described herein may be introduced into the host cell using any available technique known in the art. For example, the plasmid may be introduced into the host cell by lipofection, calcium phosphate transfection, DEAE-dextran mediated transfection, electroporation, transduction, sonoporation, infection and optical transfection. Suitable host cells include, but are not limited to, bacterial cells (e.g., E. coli, Bacillus subtilis, and Salmonella typhimurium), yeast cells (e.g., Saccharomyces cerevisiae and Schizosaccharomyces pombe), plant cells (e.g., Nicotiana tabacum and Gossypium hirsutum), and mammalian cells (e.g., CHO cells, and 3T3 fibroblasts, HEK 293 cells, U-2 OS cells).

In some embodiments, contacting a host cell transformed with the plasmid described herein with a test compound comprises growing the transformed host cell in the presence of the test compound for an appropriate time. under suitable culture conditions. Suitable culture conditions, including the duration of the culture, will vary depending on the cell being cultured. However, one skilled in the art can easily determine the culture conditions by following standard protocols, such as those described in the series Methods in Microbiology, Academic Press Inc. Typically, the cell culture medium may contain any of the following nutrients in appropriate amounts and combinations: salt(s), buffer(s), amino acids, glucose or other sugar(s), antibiotics, serum or serum replacement, and other components such as, but not limited to, peptide growth factors, cofactors, and trace elements. In some embodiments, the transfected host cells are grown in the presence of the compound for 15 mins, 30 mins, 1 hour, 2 hours, 4 hours, 6 hours, 8 hours, 10 hours, 12 hours, 14 hours, 16 hours, 18 hours, 20 hours, 24 hours, 30 hours, 48 hours, or 72 hours.

In some embodiments, a single transformed host cell is first isolated, cloned and expanded based on optimized responses to a control test compound and confirmed to provide sufficiently low error required for HTS campaigns. Selection of appropriate clones is aided by determining the response of the fused reporter protein of interest relative to the unfused reporter with the necessary response stability and reproducibility required for high throughput screening. Identification of useful clones is aided by additionally normalizing the fused reporter signals to the control unfused reporter which can significantly reduce the inherent error relative to measuring the response solely from the fused reporter. This reduction in error is critical for the identification of a useful clonal cell line that responds to a test compound with a large enough response relative to the response error obtained from the respective signals observed from the treated and untreated samples in order to provide a Z factor sufficient for high throughput screening. (en.wikipedia.org/wiki/Z-factor).

As used herein, “fused reporter protein signal” refers to the detectable signal of the reporter protein encoded by the nucleotide sequence that is fused to the ORF. As used herein, “unfused reporter protein signal” refers to the detectable signal of the reporter protein encoded by the nucleotide sequence that is not fused to the ORF. The fused and unfused reporter protein signals in the presence and absence of the test compound are determined using methods known in the art. Detectors such as, but not limited to, luminometers, spectrophotometers, and fluorimeters, or any other device that can detect changes in reporter protein activity can be used. Assay systems known in the art that allow for quantitation of a stable reporter signal from two reporter genes in a single sample can be used. Examples include, but are not limited to, Dual-Glo® Luciferase Assay System (Promega) that measures the activities of firefly and Renilla luciferases sequentially from a single sample.

After detecting the signals generated by the reporter proteins, the ratio of the fused reporter protein signal to unfused reporter protein signal in the presence of the test compound is compared to the ratio of the fused reporter protein signal to unfused reporter protein signal in the absence of the test compound. When the ratio of fused reporter protein signal to unfused reporter protein signal in the presence of the test compound is increased as compared to the ratio of fused reporter protein signal to unfused reporter protein signal in the absence of the test compound, the test compound is identified as a stabilizer of the protein of the interest. In contrast, when the ratio of fused reporter protein signal to unfused reporter protein signal in the presence of the test compound is decreased as compared to the ratio of fused reporter protein signal to unfused reporter protein signal in the absence of the test compound, the test compound is identified as a destabilizer of the protein of interest.

In some embodiments, the open reading frame is fused to the nucleotide sequence encoding a first reporter protein. In such embodiments, ratios of first reporter protein signal to second reporter protein signal are determined in presence and absence of the compound. The test compound is identified as a stabilizer when the ratio of the first reporter protein signal to second reporter protein signal in the presence of the test compound is increased as compared to the ratio of first reporter protein signal to second reporter protein signal in the absence of the test compound. The test compound is identified as a destabilizer when the ratio of first reporter protein signal to second reporter protein signal in the presence of the test compound is decreased as compared to the ratio of first reporter protein signal to second reporter protein signal in the absence of the test compound.

In some embodiments, the open reading frame is fused to the nucleotide sequence encoding a second reporter protein. In such embodiments, ratios of second reporter protein signal to first reporter protein signal are determined in presence and absence of the compound. The test compound is identified as a stabilizer when the ratio of the second reporter protein signal to first reporter protein signal in the presence of the test compound is increased as compared to the ratio of second reporter protein signal to first reporter protein signal in the absence of the test compound. The test compound is identified as a destabilizer when the ratio of second reporter protein signal to first reporter protein signal in the presence of the test compound is decreased as compared to the ratio of second reporter protein signal to first reporter protein signal in the absence of the test compound.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

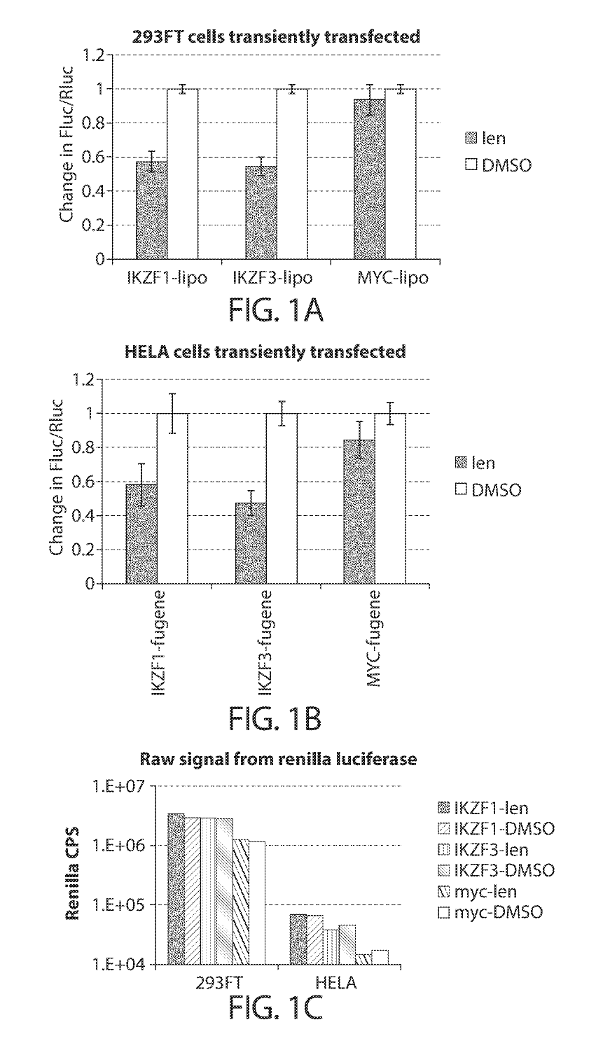

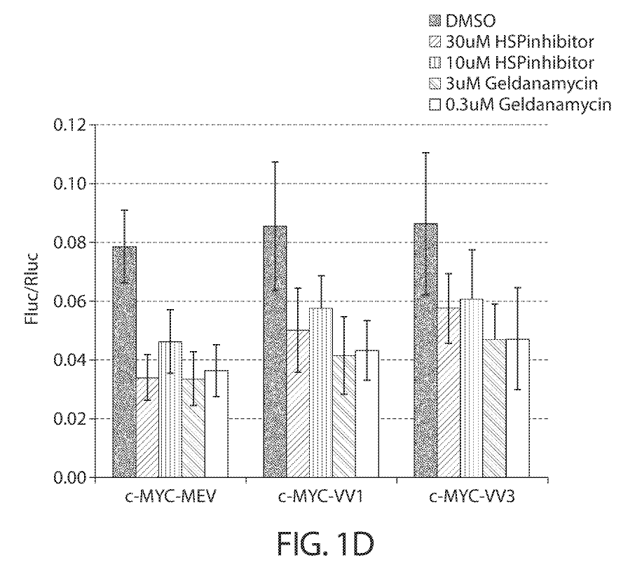

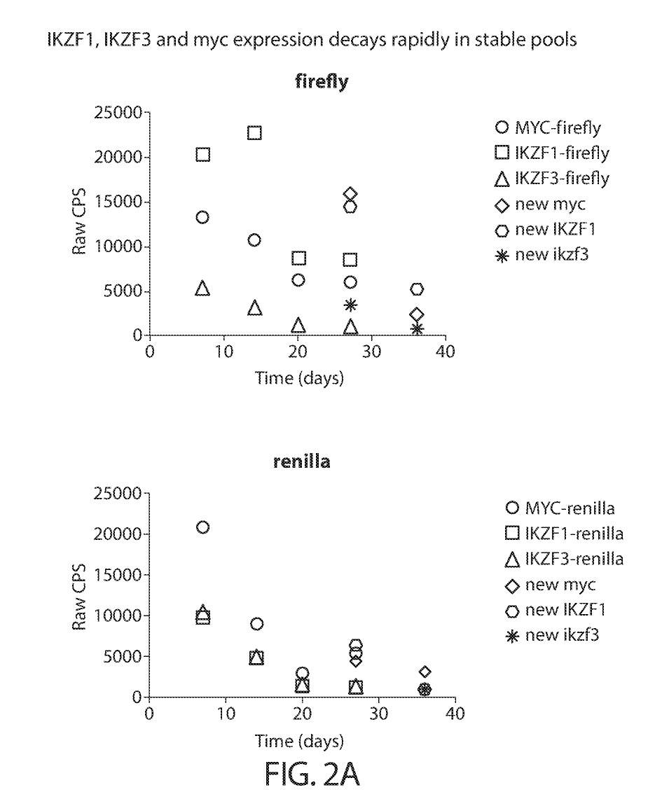

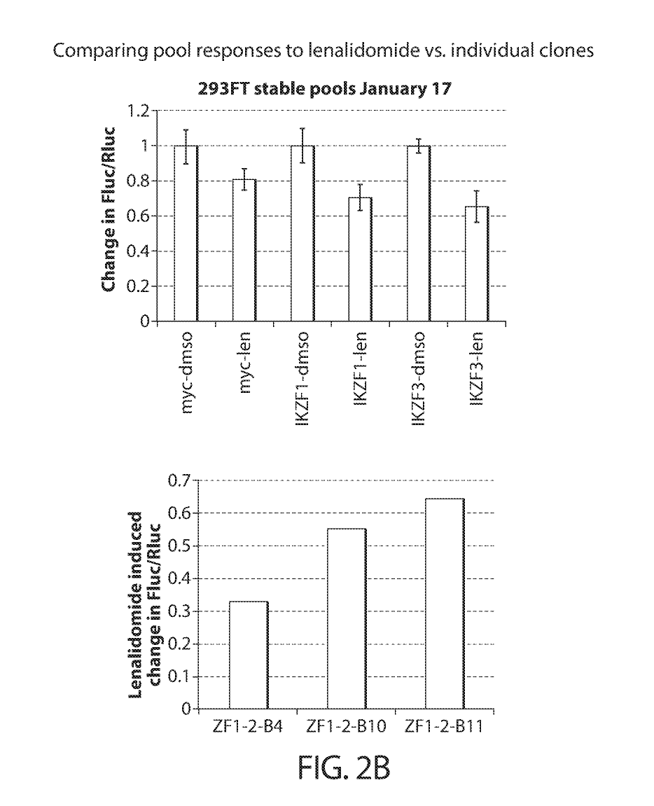

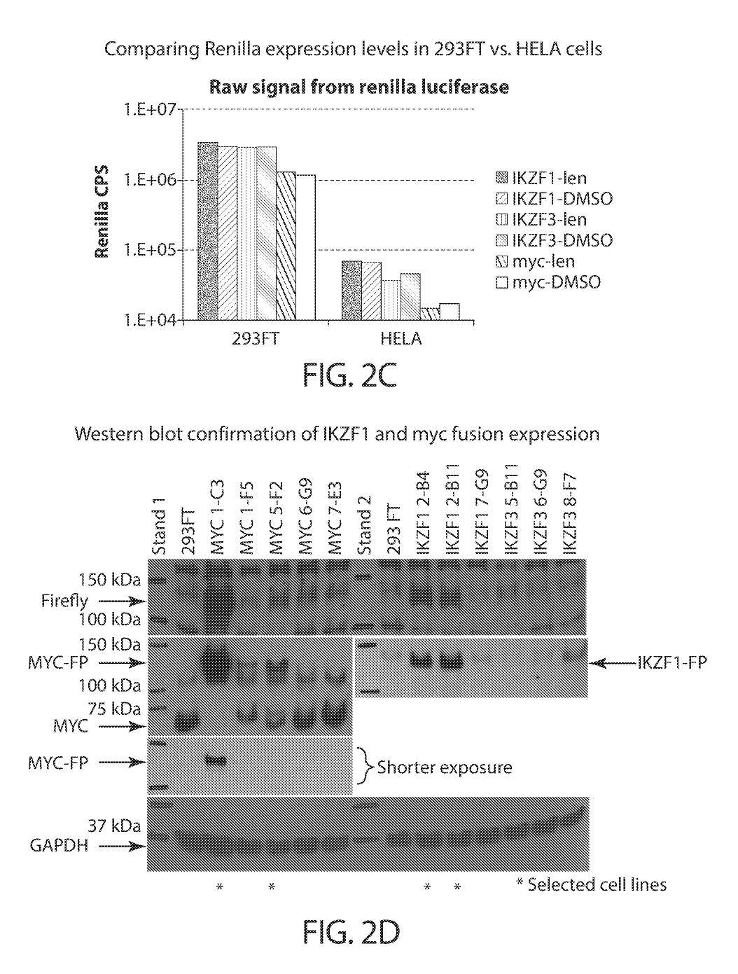

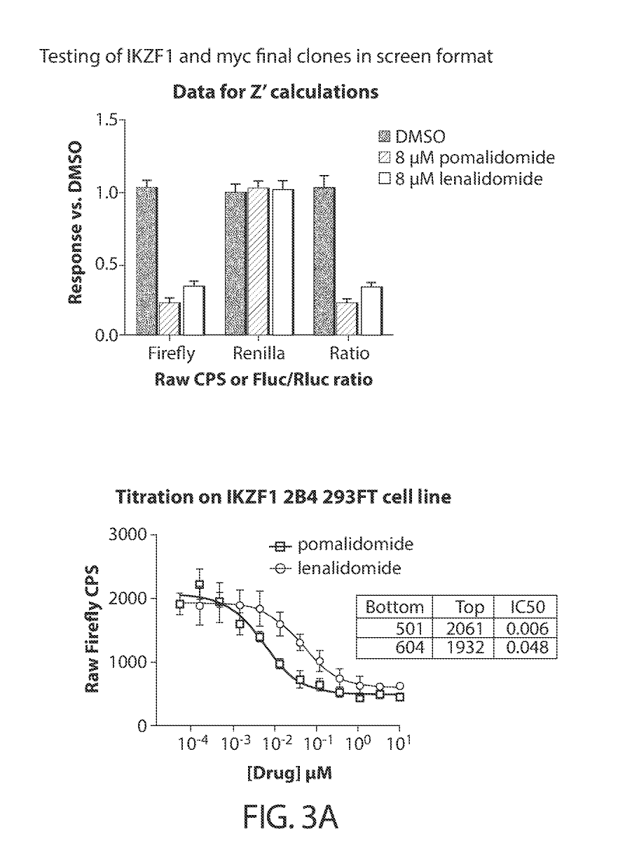

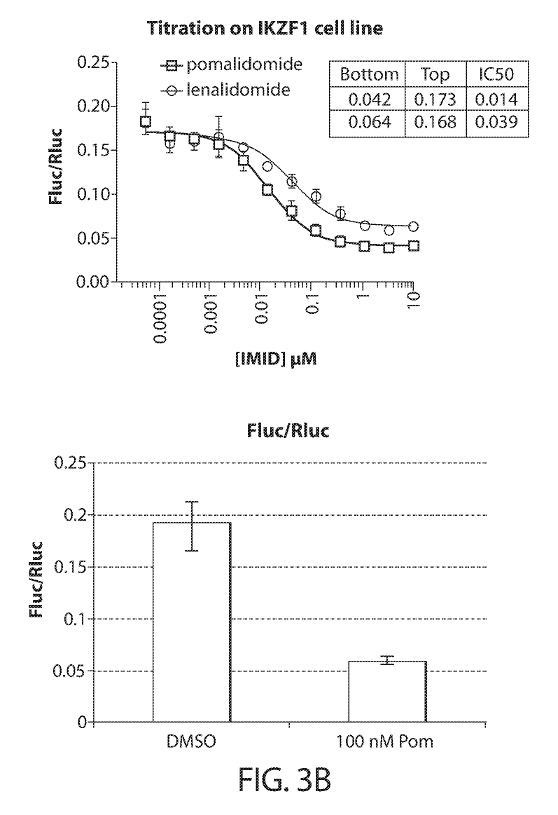

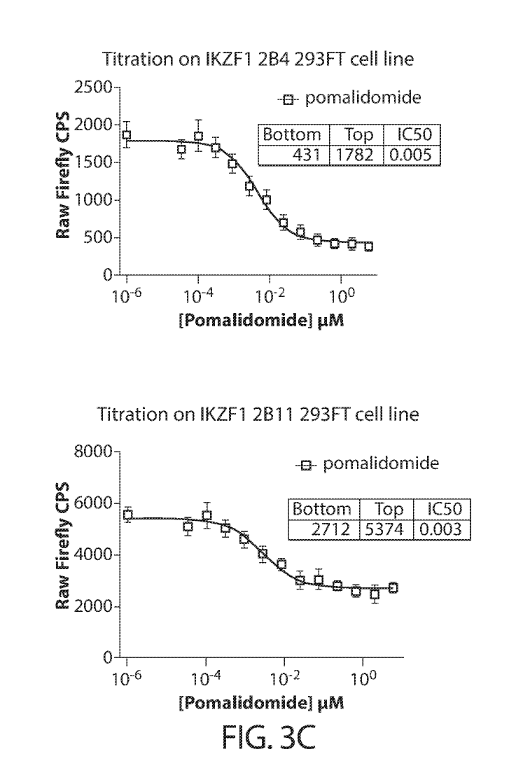

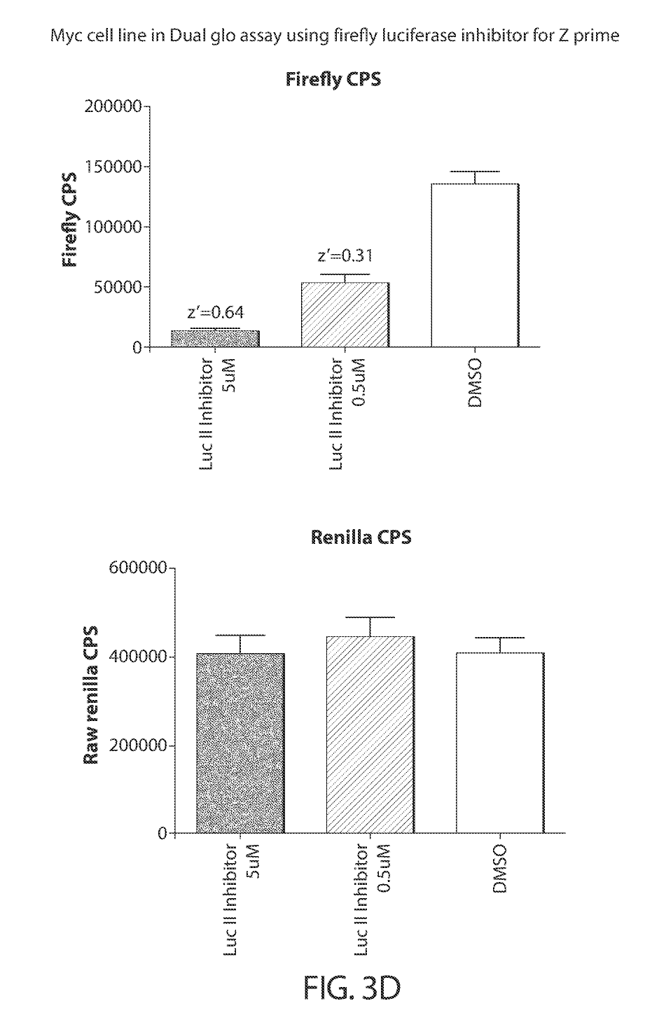

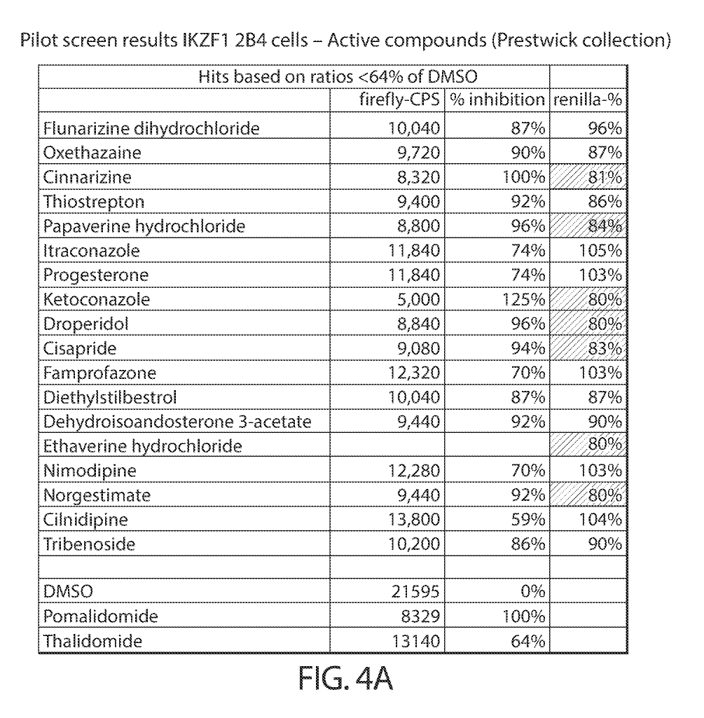

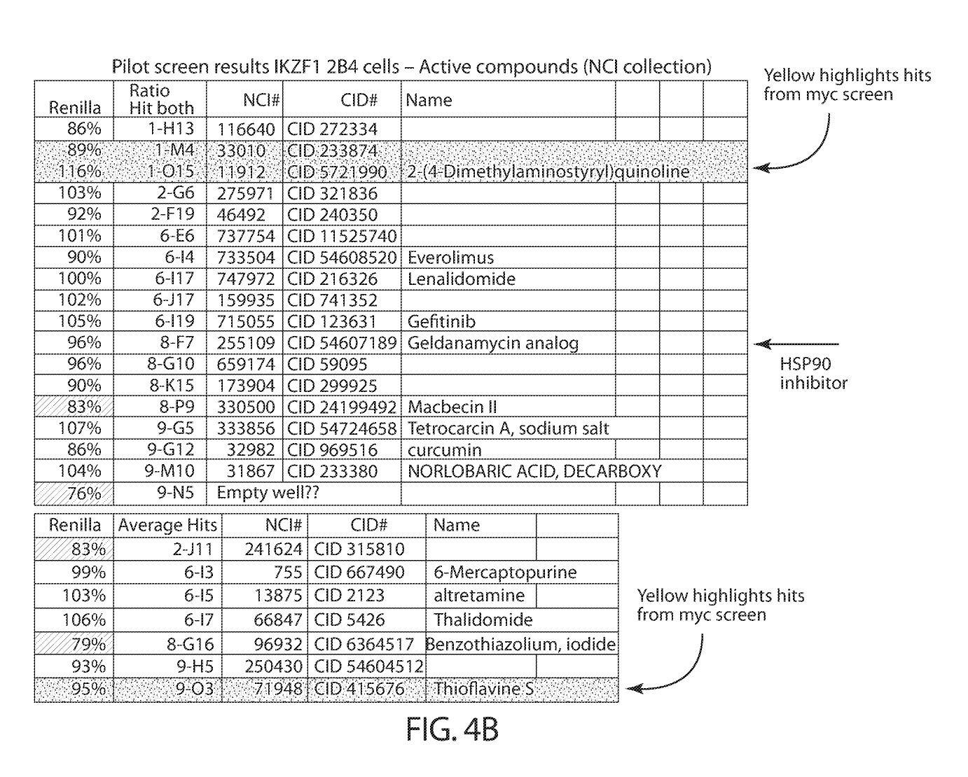

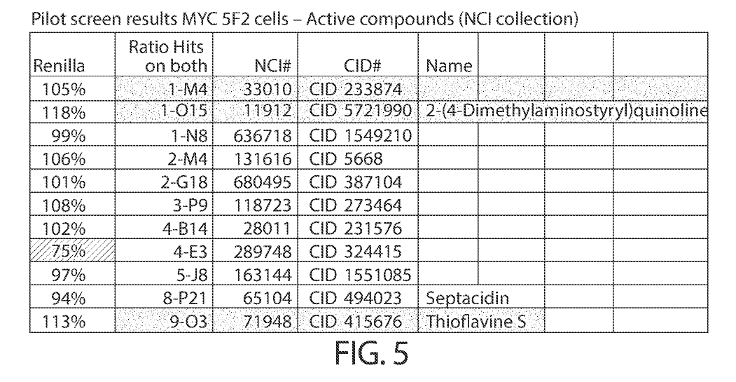

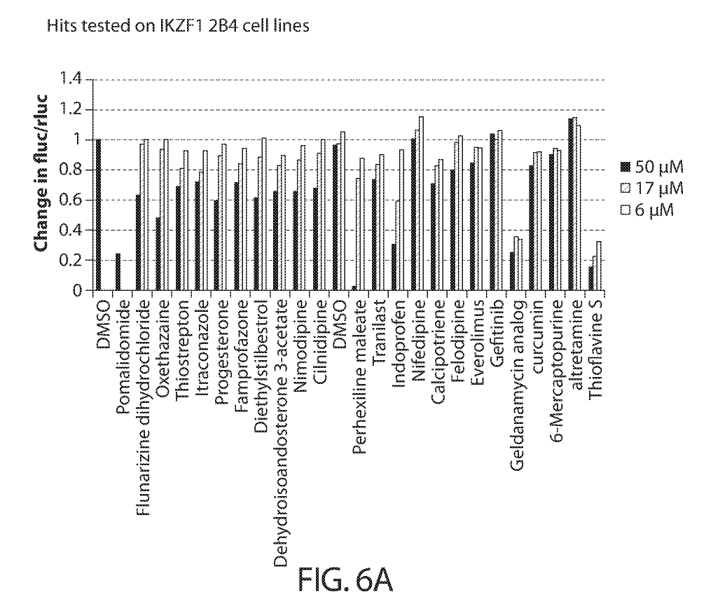

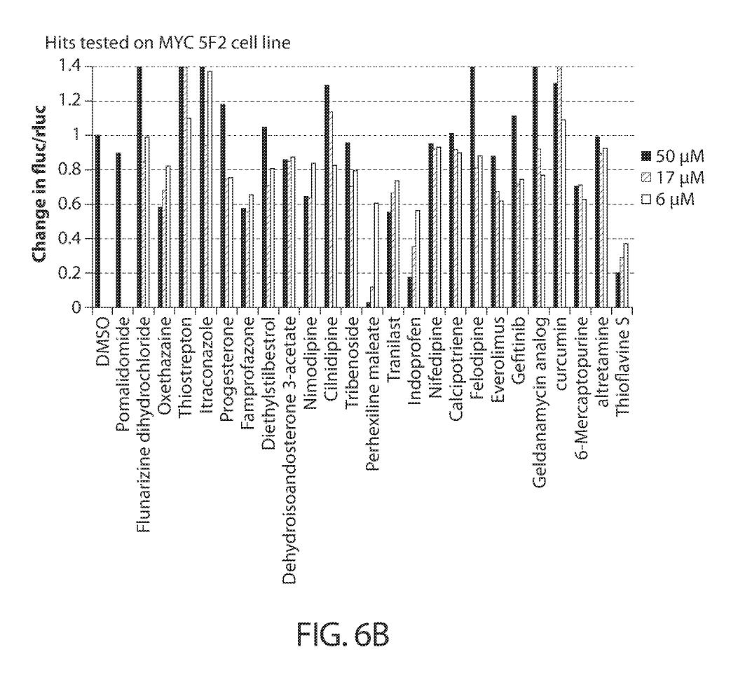

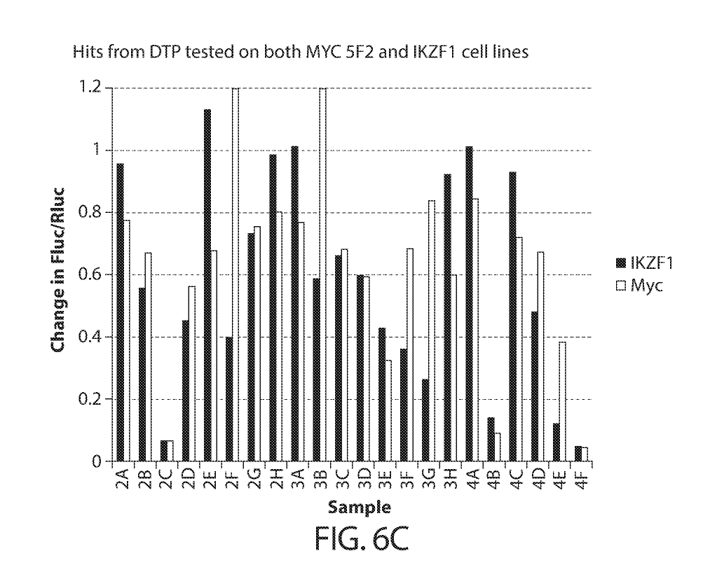

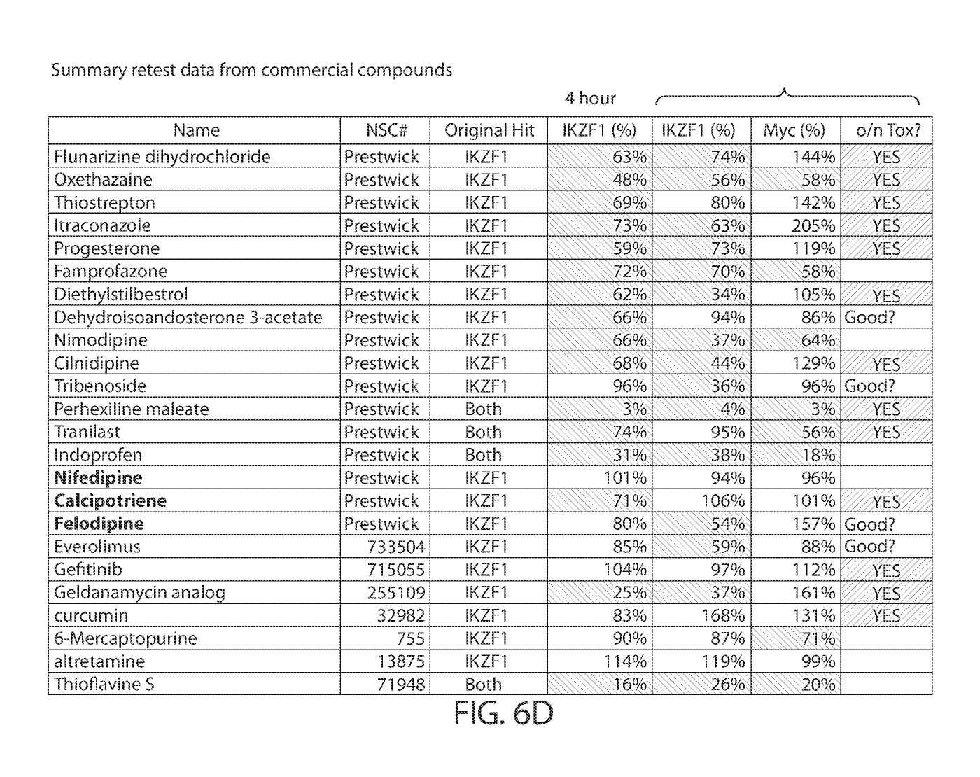

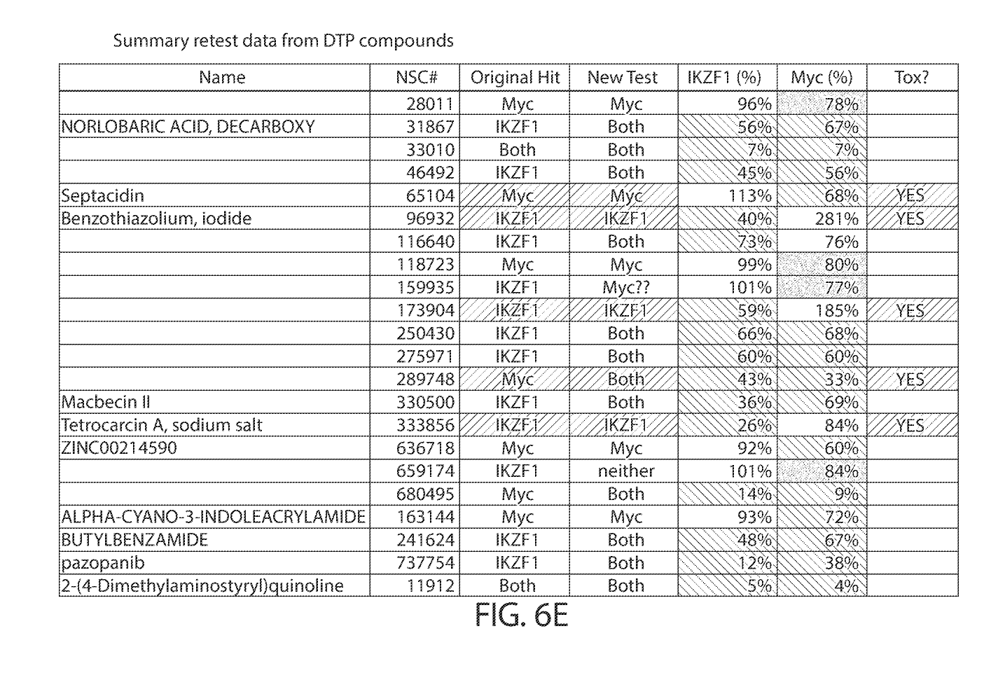

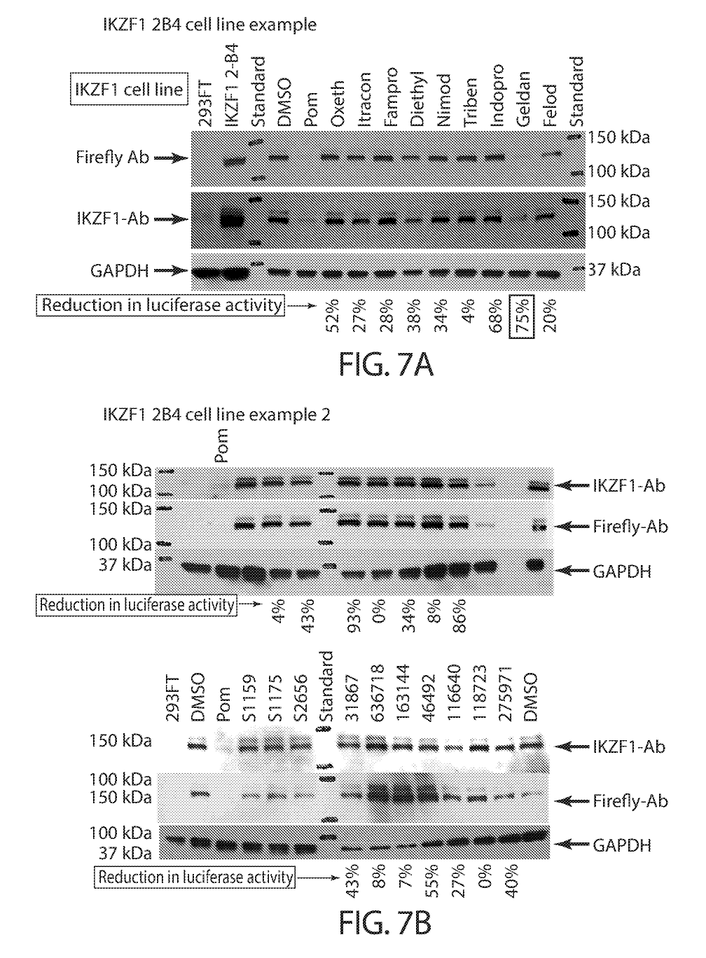

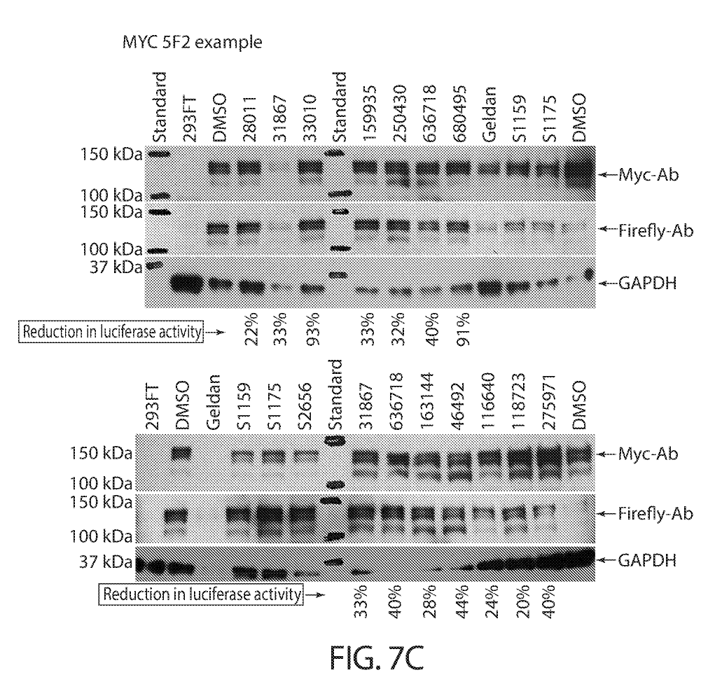

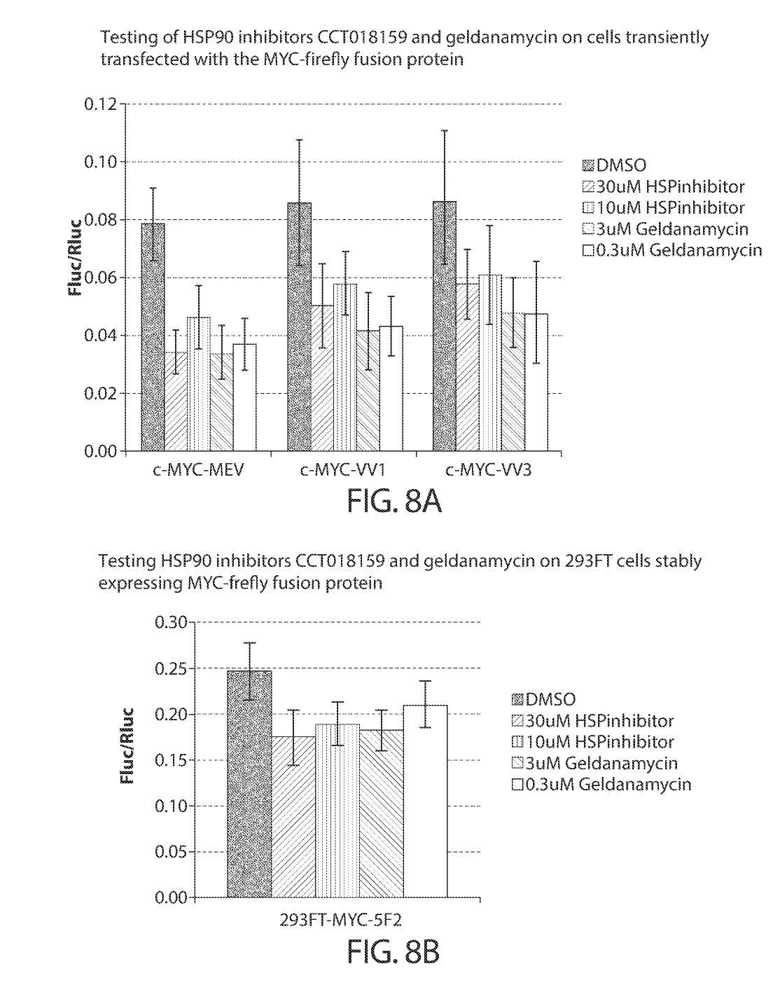

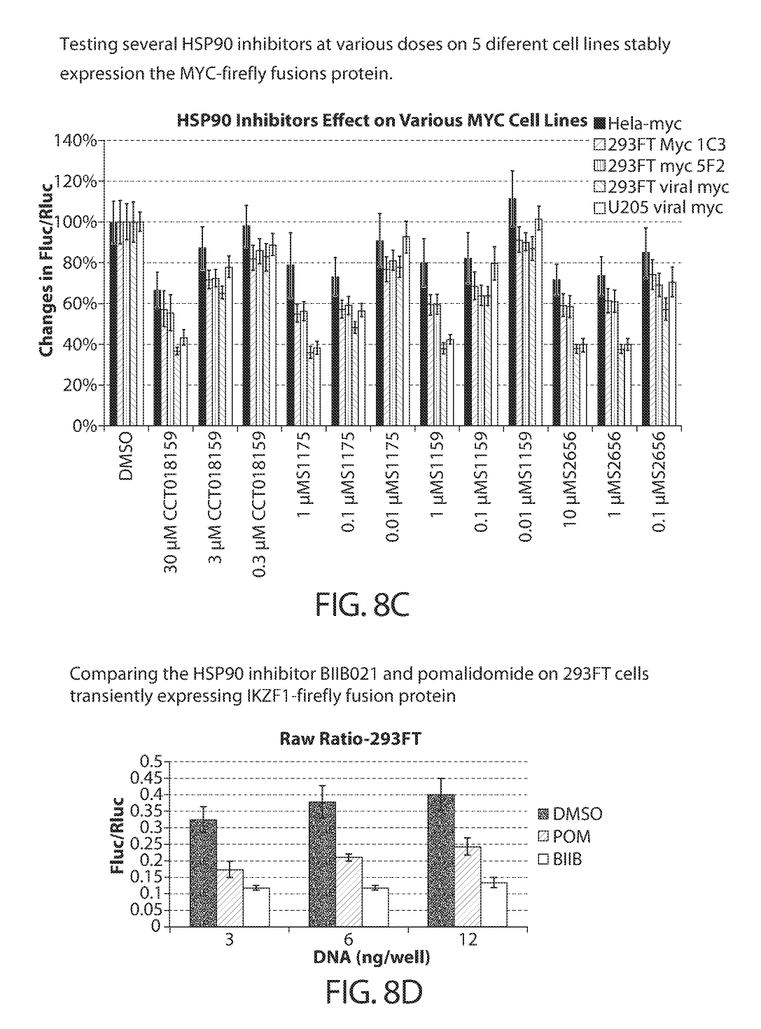

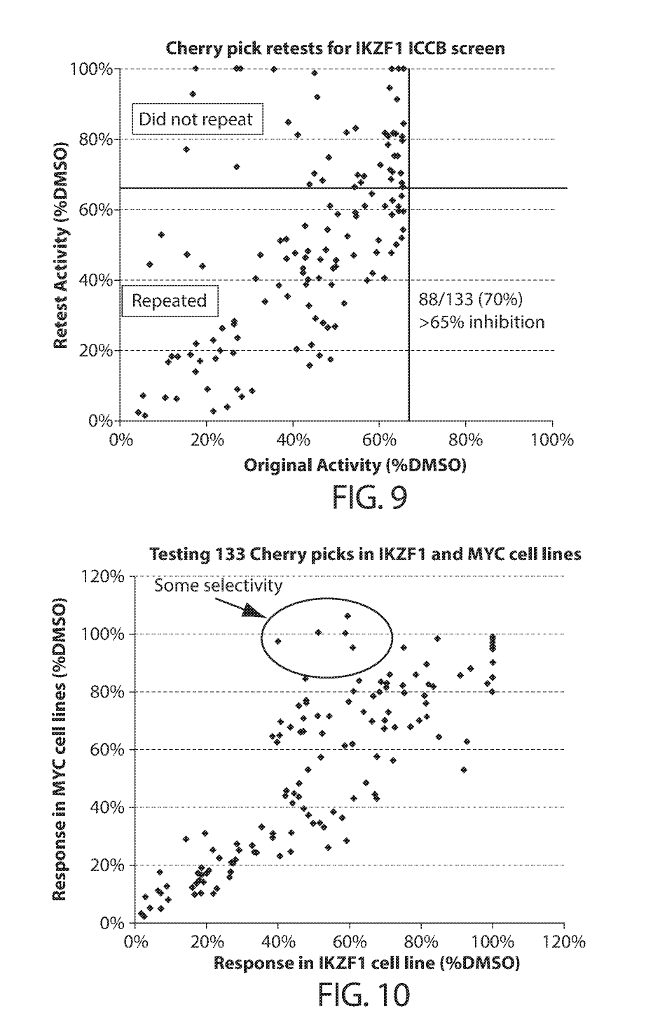

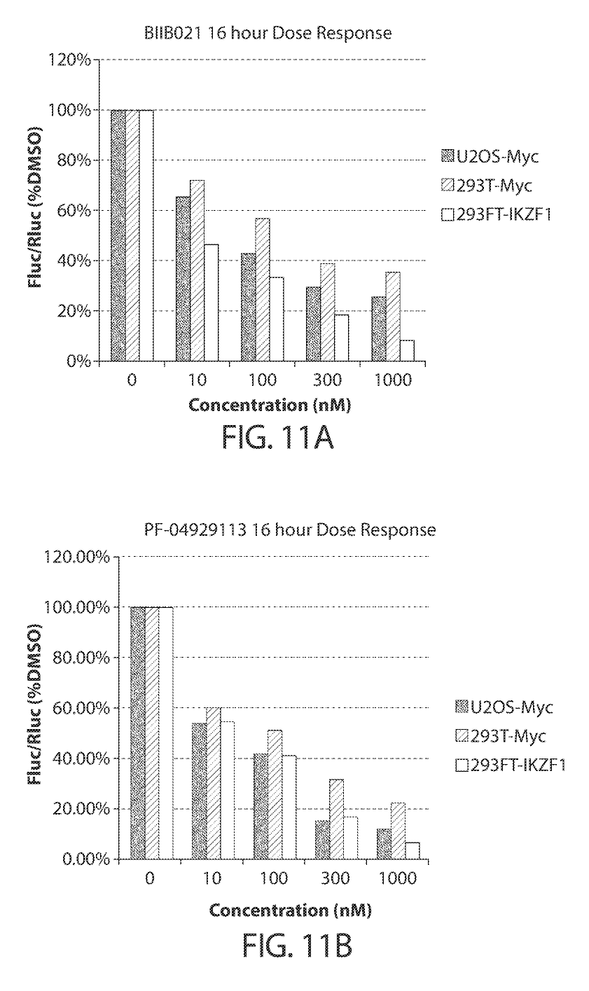

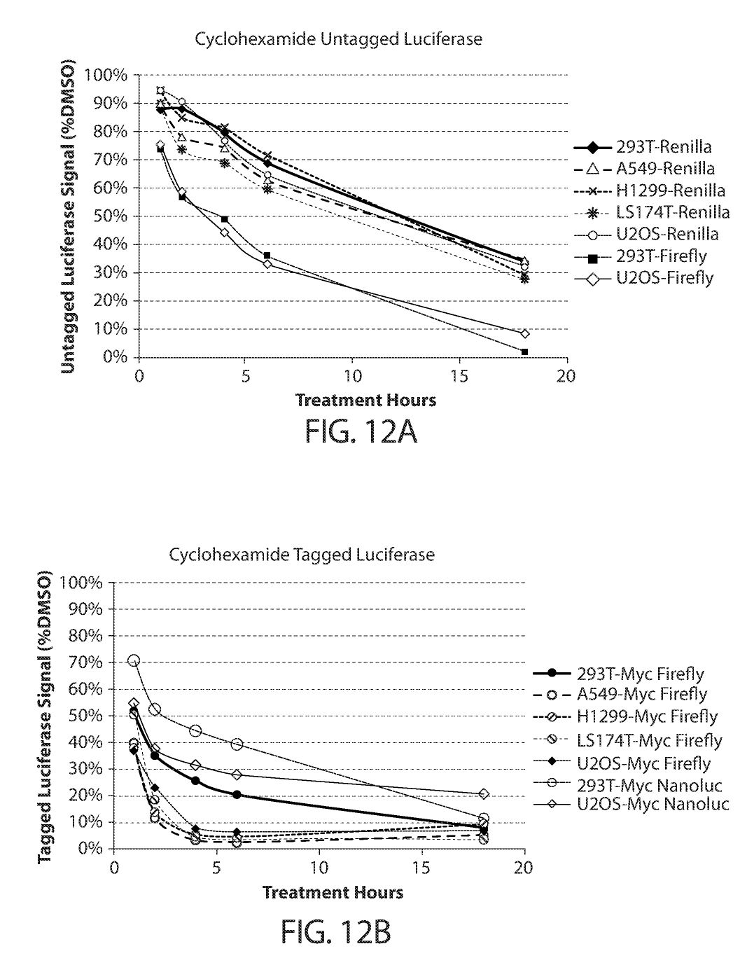

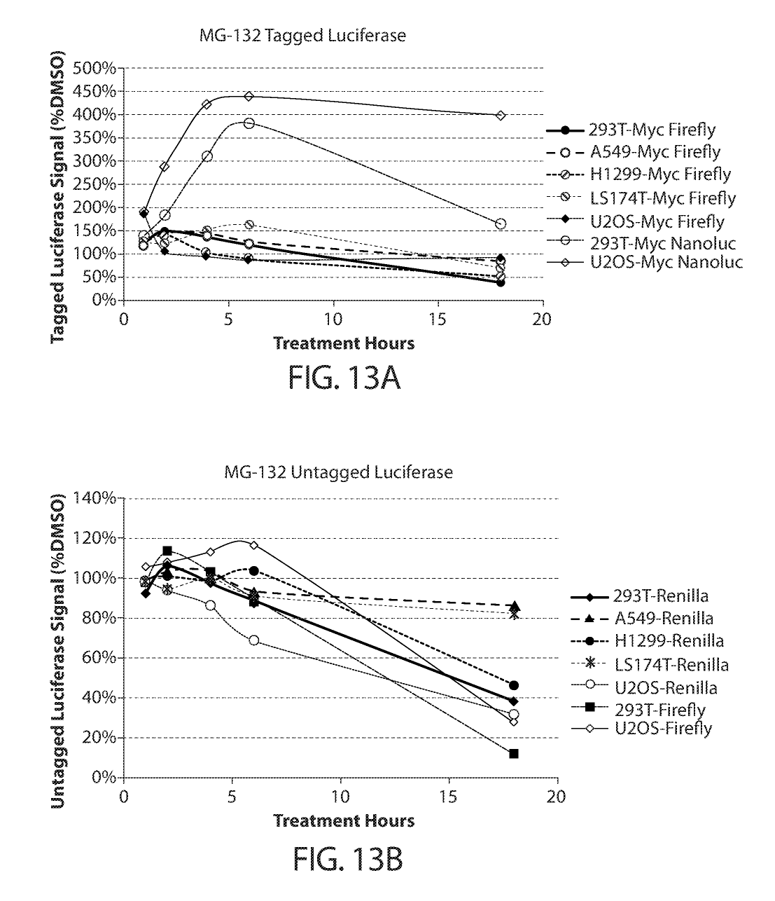

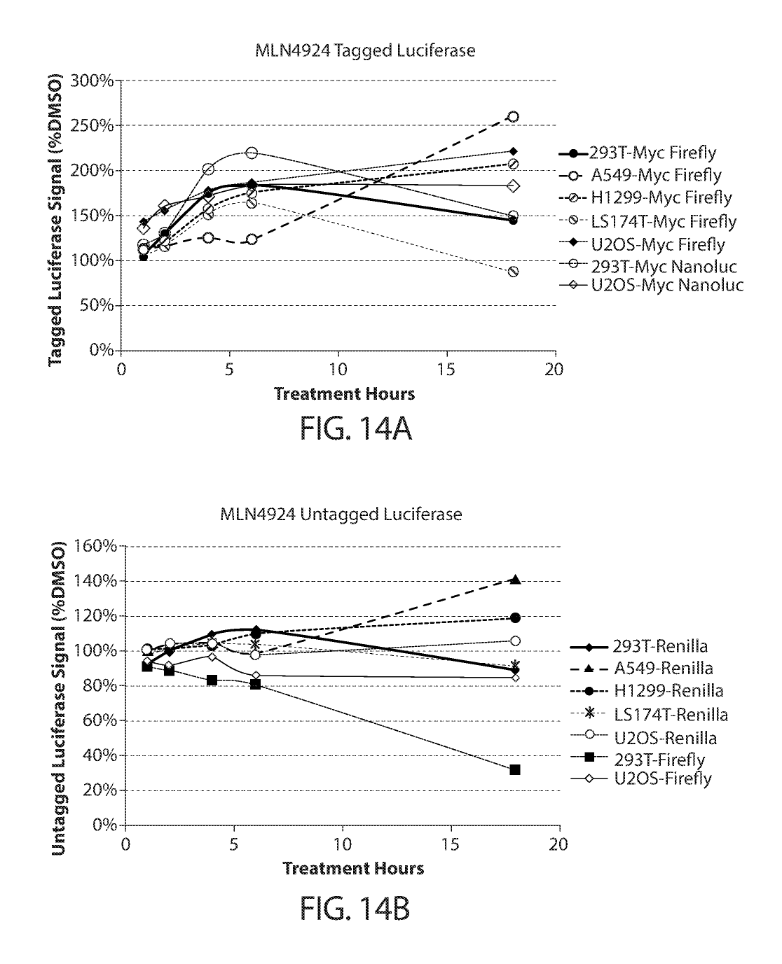

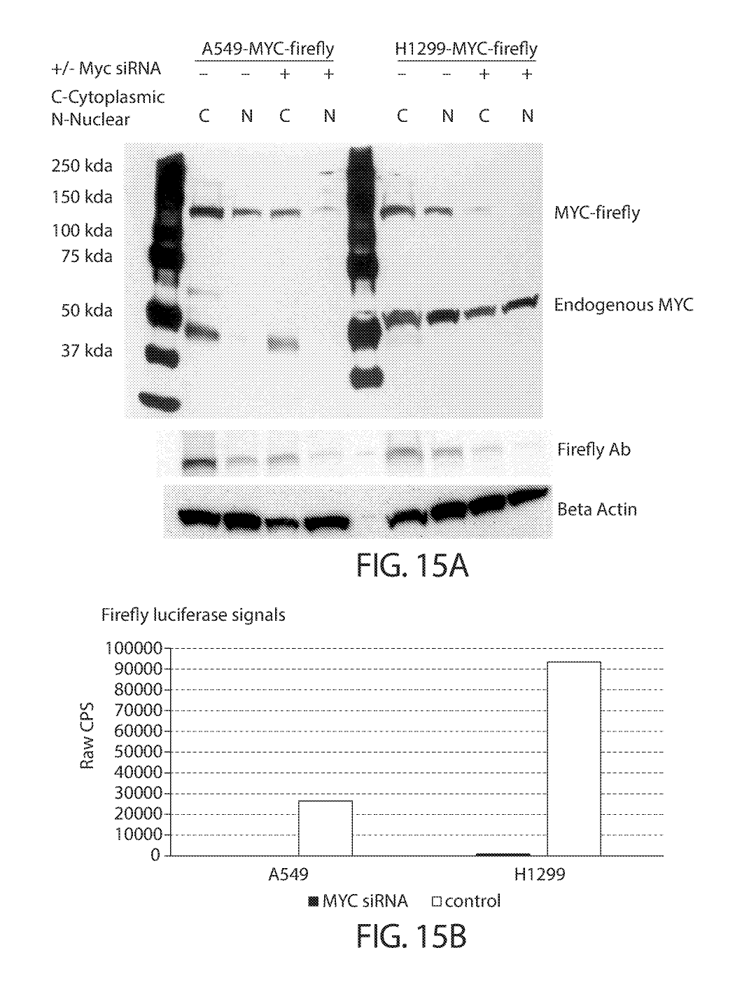

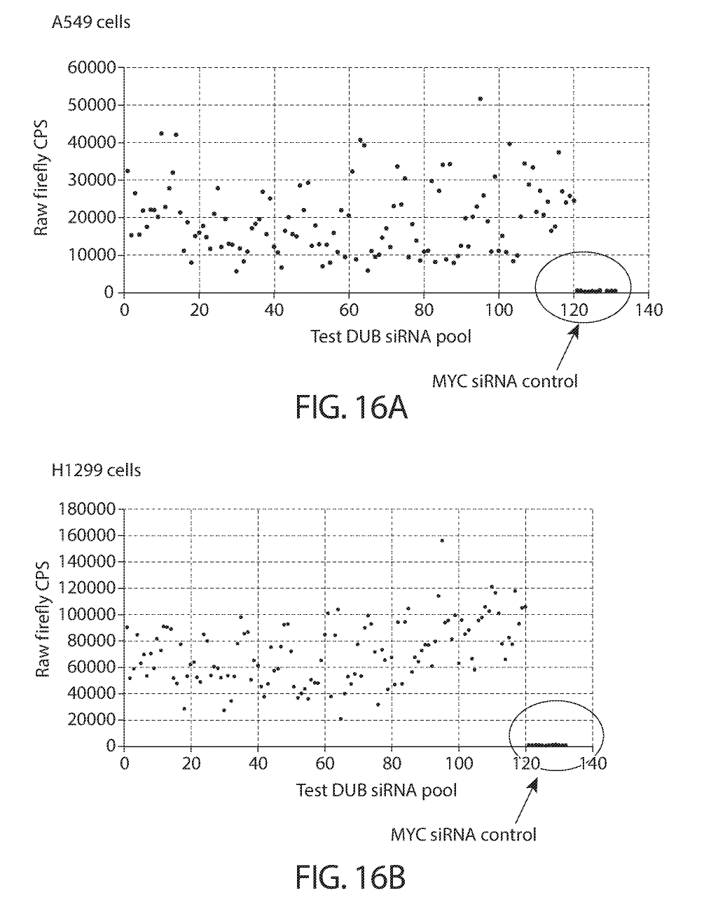

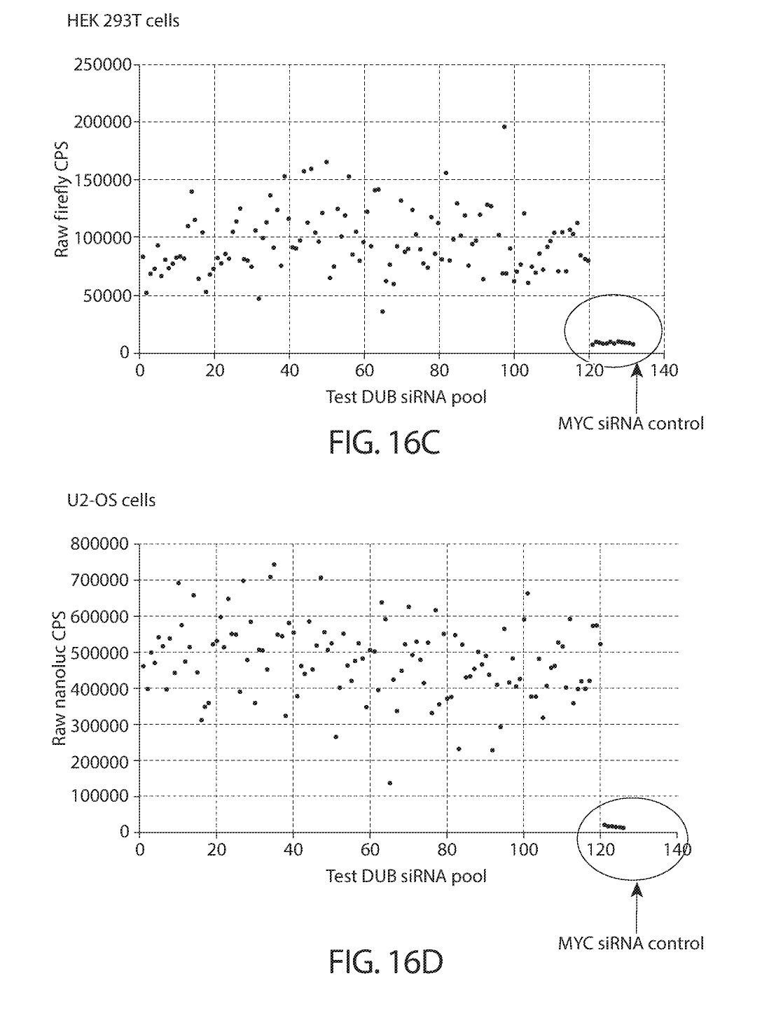

pIRIGF constructs express in 293FT and HELA cells (FIG. 1A-C) and pUG-FIRP constructs express in U-2 OS cells (FIG. 1D). Several different versions of mammalian and lentiviral plasmid constructs were tested for their ability to generate cells (e.g. 293FT, HELA, or U-2 OS cells) expressing tagged target proteins (e.g., firefly or NanoLuc tag) and co-expressing a reporter luciferase (e.g., Renilla or Firefly).

293FT and HELA cells were transfected with IKZF1-firefly, IKZF3-firefly and MYC-firefly fusion proteins and selected using puromycin and geneticin respectively. These pools were very unstable and lost signals in 10 to 30 days and generally had very small responses to IMiD's (FIG. 2A-C). Therefore, individual clones were isolated using a limited cloning strategy in 96-well plates. Surviving cells were isolated as colonies, further expanded and tested for luciferase signals and response to IMiDs. Clone 2B4 was identified as a strong responder to lenalidomide. HELA cells expressed very low levels of luciferase making isolation of HELA clones very difficult. Detection by western blots of firefly, IKZF1 and myc confirmed expression of the fusion protein and relative expression correlated with firefly luciferase signals (FIG. 2D).

Cell line clones (IKZF1-2B4, IKZF1-2B11, myc-1C3 and myc-5F2) expressing the indicated firefly fusion protein were evaluated in the dual-glo assay for reproducibility. Potency of IMiD's and relative reduction in firefly luciferase signals confirmed the expected responses and generated data with Z′ values sufficient for screening (FIG. 3A-D).

Pilot screen results for IKZF1 2B4 cells—Active compounds (Prestwick collection and NCI collection are shown in FIGS. 4A and 4B.

Pilot screen results for MYC 5F2 cells—Active compounds NCI collection are shown in FIG. 5.

The hits tested on IKZF1 2B4 and MYC 5F2 cell lines were confirmed (FIG. 6A-C). Summary retest data from commercial compounds and from DTP compounds is shown in FIG. 6D-E.

FIG. 7 shows confirmation data using Western blots. IKZF1 2B4 cell line examples (FIG. 7A-B), MYC 5F2 example (FIG. 7C)

FIG. 8 shows further evaluations of HSP90 inhibitors. FIG. 8A demonstrates testing of HSP 90 inhibitors CCT018159 and geldanamycin on cells transiently transfected with the MYC-firefly fusion protein. FIG. 8B shows testing of HSP 90 inhibitors CCT018159 and geldanamycin on 293FT cells stably expressing MYC-firefly fusion protein. FIG. 8C shows testing of several HSP-90 inhibitors at various doses on 5 different cell lines stably expressing the MYC-firefly fusions protein. FIG. 8D compares the HSP90 inhibitor BIIB021 and pomalidomide on 293FT cells transiently expressing IKZF1-firefly fusion protein.

### Example 2: Comparing Activity in IKZF1 vs. MYC Cell Lines

### Screening Campaign at HMS Screening Facility ICCB

Cherry pick retests for IKZF1 were screened at ICCB (FIG. 9). Of the 44,460 compounds, 0.6% of the compounds that were screened had a hit rate based on greater than 35% decrease in Fluc/Rluc compared to plate average. 0.3% of the compounds were cherry picked. 81% (108/133) of the hits repeated with greater than 25% decrease in Fluc/Rluc compared to DMSO control. Approximately 90% (97/108) were hit in both cell lines. There was an 11 >25% difference in activity for IKZF1 vs. MYC. All of the above results were moderate or weak hits.

The results of the ICCB cherry picks IKZF1 vs. MYC selectivity comparison show that the majority of hits in the IKZF1 cell line screen were also active in the counter screen assay suggesting a nonspecific mechanism (FIG. 10). Five compounds were inactive in the counter screen, but still reduced IKZF1 luciferase signal more than 35%, showing some selectivity.

### Dose Response for HSP90 Inhibitors

Two HSP90 inhibitors, BIIB021 (FIG. 11A) and PF-04929113 (FIG. 11B), show similar activity in the 293FT IKZF1 cell line compared to the counter screen 293FT cell line expressing MYC-firefly:renilla and U2OS cell line expressing MYC-firefly:renilla suggesting a mechanism nonselective for IKZF1. Knockdown of protein levels were confirmed by western blot indicating that the luciferase reporter system is accurately reflecting fusion protein reduction.

### Stability Time Course for 7 MYC-Luciferase Fusion Cell Lines

Seven cell lines were used to measure the half life of the luciferases after blocking all protein synthesis with cyclohexamide. The decay observed for both fused luciferases (MYC-firefly and MYC-nanoluc; FIG. 12B) were compared to the decay observed for the unfused luciferases (renilla and firefly; FIG. 12A) using both the MYC-firefly:renilla and MYC-nanoluc:firefly cell lines 293T, U2OS and the MYC-firefly:renilla cell lines A549, H1299 and LS174T. As expected, the half-life of untagged firefly (approximately 4 hours) is shorter than the untagged renilla (approximately 12 hours) since it contains a PEST domain. MYC nanoluc half life of approximately 2 hours is longer than MYC-firefly half-life of less than 1 hour and closer to the half-life of untagged firefly. The balanced half-life of MYC-nanoluc and untagged firefly should reduce the number of artefact hits.

Seven MYC-luciferase reporter cell lines were used to measure changes in MYC-luciferase expression after blocking the proteasome with MG132. The expression of the unfussed renilla and firefly were unchanged for about 6 hours but decreased after 18 hours to a variable extent among cell lines (FIG. 13B). All cell lines showed at least a 50% increase in MYC-luciferase fusion protein, but with different time course. The MYC-nanoluc demonstrated about 4-fold increase in luciferase signals suggesting a larger portion of the these fusion proteins are degraded by the proteasome (FIG. 13A).

Seven MYC-luciferase reporter cell lines were used to measure changes in MYC-luciferase expression after inhibition of ubiquitin dependent proteolysis with the neddylation inhibitor MLN-4924 (FIG. 14A-B). The expression of the unfussed renilla and firefly were minimally affected except for the 293T MYC-firefly:renilla cell line. All cell lines showed at least a 50% increase in MYC-luciferase fusion protein, typically peaking after 6 hours of treatment. These results demonstrate that ubiquitin dependent proteolysis is at least partially responsible for the stability of the MYC-fusion proteins in all 7 cell lines.

A549 and H1299 Cell Lines Expressing MYC-Firefly after siMYC Knockdown

siRNA was used to knockdown the MYC-firefly luciferase fusion protein in the A549 and H1299 cell lines using 48 hour treatment with siRNA directed to MRC mRNA. The reduction in fusion protein, as observed by western blotting with MYC and firefly directed antibodies (FIG. 15A), is comparted to the decrease in luciferase signals (FIG. 15B). MYC antibody also detects the decrease in endogenous MYC. A prominent MYC-NICK band is observed in the A549 cells.

siGENOME siRNA Library

FIG. 16A-D shows screening results from a commercial library of siRNA's directed to the family of DUB enzymes with A549, H1299, and HEK293T cells expressing MYC-firefly and U2OS cells expressing MYC-nanoluc.

### REFERENCES

1. R. Martiniani, V. Di Loreto, C. Di Sano, A. Lombardo, A. M. Liberati, Biological activity of lenalidomide and its underlying therapeutic effects in multiple myeloma. Adv Hematol 2012, 842945 (2012).
2. E. Terpos, N. Kanellias, D. Christoulas, E. Kastritis, M. A. Dimopoulos, Pomalidomide: a novel drug to treat relapsed and refractory multiple myeloma. OncoTargets and therapy 6, 531 (2013).
3. Y. X. Zhu, K. M. Kortuem, A. K. Stewart, Molecular mechanism of action of immune-modulatory drugs thalidomide, lenalidomide and pomalidomide in multiple myeloma. Leukemia & lymphoma 54, 683 (April, 2013).
4. T. Ito et al., Identification of a primary target of thalidomide teratogenicity. Science 327, 1345 (Mar. 12, 2010).
5. A. Lopez-Girona et al., Cereblon is a direct protein target for immunomodulatory and antiproliferative activities of lenalidomide and pomalidomide. Leukemia 26, 2326 (November, 2012).
6. L. H. Zhang et al., Lenalidomide efficacy in activated B-cell-like subtype diffuse large B-cell lymphoma is dependent upon IRF4 and cereblon expression. Br J Haematol 160, 487 (February, 2013).
7. Y. Yang et al., Exploiting synthetic lethality for the therapy of ABC diffuse large B cell lymphoma. Cancer Cell 21, 723 (Jun. 12, 2012).
8. Y. X. Zhu et al., Cereblon expression is required for the antimyeloma activity of lenalidomide and pomalidomide. Blood 118, 4771 (Nov. 3, 2011).
9. A. Broyl et al., High cereblon expression is associated with better survival in patients with newly diagnosed multiple myeloma treated with thalidomide maintenance. Blood 121, 624 (Jan. 24, 2013).
10. D. Heintel et al., High expression of cereblon (CRBN) is associated with improved clinical response in patients with multiple myeloma treated with lenalidomide and dexamethasone. Br J Haematol 161, 695 (June, 2013).
11. G. J. Zhang et al., Bioluminescent imaging of Cdk2 inhibition in vivo. Nat Med 10, 643 (June, 2004).
12. M. Safran et al., Mouse model for noninvasive imaging of HIF prolyl hydroxylase activity: assessment of an oral agent that stimulates erythropoietin production. Proc Natl Acad Sci USA 103, 105 (Jan. 3, 2006).
13. H. C. Yen, Q. Xu, D. M. Chou, Z. Zhao, S. J. Elledge, Global protein stability profiling in mammalian cells. Science 322, 918 (Nov. 7, 2008).
14. T. A. Soucy et al., An inhibitor of NEDD8-activating enzyme as a new approach to treat cancer. Nature 458, 732 (Apr. 9, 2009).
15. M. Ohh et al., An intact NEDD8 pathway is required for Cullin-dependent ubiquitylation in mammalian cells. EMBO reports 3, 177 (February, 2002).
16. L. Cong et al., Multiplex genome engineering using CRISPR/Cas systems. Science 339, 819 (Feb. 15, 2013).
17. P. Mali et al., RNA-guided human genome engineering via Cas9. Science 339, 823 (Feb. 15, 2013).
18. M. Merkenschlager, Ikaros in immune receptor signaling, lymphocyte differentiation, and function. FEBS Lett 584, 4910 (Dec. 15, 2010).
19. E. C. Thompson et al., Ikaros DNA-binding proteins as integral components of B cell developmental-stage-specific regulatory circuits. Immunity 26, 335 (March, 2007).
20. I. Ferreiros-Vidal et al., Genome-wide identification of Ikaros targets elucidates its contribution to mouse B-cell lineage specification and pre-B-cell differentiation. Blood 121, 1769 (Mar. 7, 2013).
21. S. Monticelli, F. Sallusto, Negative regulators take center stage. Nature immunology 13, 719 (August, 2012).
22. L. A. Garraway, W. R. Sellers, Lineage dependency and lineage-survival oncogenes in human cancer. Nat Rev Cancer 6, 593 (August, 2006).
23. S. P. Shah et al., Mutation of FOXL2 in granulosa-cell tumors of the ovary. N Engl J Med 360, 2719 (Jun. 25, 2009).
24. P. Kastner et al., Function of Ikaros as a tumor suppressor in B cell acute lymphoblastic leukemia. American journal of blood research 3, 1 (2013).
25. F. J. Quintana et al., Aiolos promotes TH17 differentiation by directly silencing 112 expression. Nature immunology 13, 770 (August, 2012).
26. N. Avitahl et al., Ikaros sets thresholds for T cell activation and regulates chromosome propagation. Immunity 10, 333 (March, 1999).
27. J. Laubach, P. Richardson, K. Anderson, Multiple myeloma. Annu Rev Med 62, 249 (2011).
28. P. G. Richardson et al., Multicenter, phase I, dose-escalation trial of lenalidomide plus bortezomib for relapsed and relapsed/refractory multiple myeloma. J Clin Oncol 27, 5713 (Dec. 1, 2009).
29. G. R. Crabtree, S. L. Schreiber, Three-part inventions: intracellular signaling and induced proximity. Trends Biochem Sci 21, 418 (November, 1996).
30. M. R. Campanero, E. K. Flemington, Regulation of E2F through ubiquitin-proteasome-dependent degradation: stabilization by the pRB tumor suppressor protein. Proc Natl Acad Sci USA 94, 2221 (Mar. 18, 1997).
31. D. Wiederschain et al., Single-vector inducible lentiviral RNAi system for oncology target validation. Cell Cycle 8, 498 (Feb. 1, 2009).
32. C. J. Ott et al., BET bromodomain inhibition targets both c-Myc and IL7R in high-risk acute lymphoblastic leukemia. Blood 120, 2843 (Oct. 4, 2012).
33. K. Kondo, J. Klco, E. Nakamura, M. Lechpammer, W. G. Kaelin, Inhibition of HIF is necessary for tumor suppression by the von Hippel-Lindau protein. Cancer Cell 1, 237 (April, 2002).
34. J. Loven et al., Selective inhibition of tumor oncogenes by disruption of super-enhancers. Cell 153, 320 (Apr. 11, 2013).

We claim:

1. A method to identify a test compound that stabilizes or destabilizes a protein of interest, the method comprising:
(i) contacting a transformed host cell comprising a DNA plasmid with a test compound, wherein the DNA plasmid comprises in operable linkage:
(a) a promoter;
(b) a first internal ribosomal entry site (IRES);
(c) a nucleotide sequence encoding a first reporter protein;
(d) a second IRES; and
(e) a nucleotide sequence encoding a second reporter protein,
wherein an open reading frame (ORF) is fused to the nucleotide sequence encoding a first reporter protein or to the nucleotide sequence encoding a second reporter protein and wherein said open reading frame codes for a protein of interest;
(ii) determining ratios of fused reporter protein signal to unfused reporter protein signal in presence and absence of the test compound; and
(iii) identifying said test compound as a stabilizer when the ratio of fused reporter protein signal to unfused reporter protein signal in the presence of the test compound is increased as compared to the ratio of fused reporter protein signal to unfused reporter protein signal in the absence of the test compound, and identifying said test compound as a destabilizer when the ratio of fused reporter protein signal to unfused reporter protein signal in the presence of the test compound is decreased as compared to the ratio of fused reporter protein signal to unfused reporter protein signal in the absence of the test compound.

2. The method of claim 1, wherein said first and second reporter proteins have distinguishable detectable reporter signals.

3. The method of claim 2, wherein said first and second reporter proteins are enzyme proteins having distinguishable signals generated from their products.

4. The method of claim 2, wherein said first and second reporter proteins are bioluminescent proteins having distinguishable bioluminescence signals.

5. The method of claim 2, wherein said first and second reporter proteins are fluorescent proteins having distinguishable fluorescence signals.

6. The method of claim 4, wherein the first and second reporter proteins are selected from the group consisting of renilla luciferase (Rluc) and firefly luciferase (FLuc).

7. The method of claim 5, wherein the first and second reporter proteins are selected from the group consisting of green fluorescence protein and red fluorescence protein.

8. The method of claim 1, wherein the promoter is a eukaryotic promoter or a synthetic promoter.

9. The method of claim 8, wherein the promoter comprises cytomegalovirus (CMV) promoter.

10. The method of claim 1, wherein said ORF is derived from an ORFeome of an organism.

11. The method of claim 1, wherein said ORF encodes an oncoprotein.

12. The method of claim 11, wherein said oncoprotein is selected from the group consisting of MYC, Ikaros family zinc finger protein 1 (IKZF1), Ikaros family zinc finger protein 3 (IKZF3), Interferon regulatory factor 4 (IRF4), mutant p53, N-Ras, c-Fos, and c-Jun.

13. The method of claim 1, wherein contacting the transformed host cell comprising the DNA plasmid with the test compound comprises growing the transformed host cell in the presence of the test compound for an appropriate time.

14. The method of claim 1, wherein the protein of interest is a transcription factor.

15. The method of claim 1, wherein the transcription factor is selected from the group consisting of STAT family transcription factor, FOS/JUN, NF KB, HIV-TAT, and E2F family transcription factor.

16. The method of claim 1, wherein the ORF is fused to a 5′ end of the nucleotide sequence encoding the first reporter protein or to a 5′ end of the nucleotide sequence encoding the second reporter protein.

17. The method of claim 1, wherein the ORF is fused to a 3′ end of the nucleotide sequence encoding the first reporter protein or to a 3′ end of the nucleotide sequence encoding the second reporter protein.

18. The method of claim 1, wherein the test compound is from a compound library and the method is performed as part of a compound screening.

19. The method of claim 18, wherein the compound library comprises synthesized organic molecules, naturally occurring organic molecules, peptides, polypeptides, or nucleic acid molecules.

20. A method to identify a test compound that stabilizes or destabilizes an oncoprotein of interest, the method comprising:
(i) contacting a transformed host cell comprising a DNA plasmid with a test compound, wherein the DNA plasmid comprises in operable linkage:
(a) a cytomegalovirus (CMV) promoter;
(b) a first internal ribosomal entry site (IRES);
(c) a nucleotide sequence encoding a renilla luciferase (Rluc) protein;
(d) a second IRES; and
(e) a nucleotide sequence encoding a firefly luciferase (FLuc) protein,
wherein an open reading frame (ORF) encoding an oncoprotein of interest is fused to the nucleotide sequence encoding the FLuc protein;
(ii) determining ratios of FLuc protein signal to Rluc protein signal in the presence and absence of the test compound; and
(iii) identifying said test compound as a stabilizer when the ratio of FLuc protein signal to Rluc protein signal in the presence of the test compound is increased as compared to the ratio of FLuc protein signal to Rluc protein signal in the absence of the test compound, and identifying said test compound as a destabilizer when the ratio of FLuc protein signal to Rluc protein signal in the presence of the test compound is decreased as compared to the ratio of FLuc protein signal to Rluc protein signal in the absence of the test compound.
